(19) United States
(12) Patent Application Publication
(10) Pub. No.: US 2006/0084990 A1
(43) Pub. Date: Apr. 20, 2006

(54) Dual anchor spinal implant apparatus

(76) Inventors: Jose Gournay, Dammartin en Goele (FR); Gaspard Demay, Paris (FR); Istvan Hovorka, Nice (FR)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 11/137,190

(22) Filed: May 25, 2005

(30) Foreign Application Priority Data
May 26, 2004 (FR) FR 04 05690

(51) Int. Cl. A61F 2/30

(52) U.S. Cl. 606/61

(57) ABSTRACT

Embodiments of devices for connecting a spinal rod to a vertebra or to two vertebrae are disclosed. In one embodiment, there is provided a first hook having a threaded protrusion for fitting a connector in which the spinal rod is inserted, a second hook, a rod for connecting the two hooks, and structure for immobilizing the ends of the hook-connecting rod on the heads of the hooks. The end of the hook-connecting rod in contact with the first hook may have an oblong aperture, and the structure for immobilizing this end on the head of the first hook may include a nut intended to cooperate with the connector. Also disclosed are embodiments of a spinal osteosynthesis system including such devices.

This application claims priority under 35 U.S.C. 119 to France Patent Application No. 04 05690, filed May 26, 2004.

### BACKGROUND

Spinal osteosynthesis devices for correcting deformations of the spine often comprise at least one rod extending along a portion of the vertebral column of the patient. It is connected to the vertebrae by devices which can take various forms. In particular, it can pass through connectors which are themselves mounted on threaded protrusions carried by plates, bone anchoring screws or hooks which are placed opposite the pedicles or on the posterior arches or transverse processes of the vertebrae.

For osteosynthesis devices or systems fitted by a posterior approach, anchoring the rod on the vertebrae by means of hooks placed on a vertebra is particularly indicated. To this end, anchoring means have been conceived in which a hook connected to the rod by a connector, as indicated above, is connected to a second hook by means of a smaller rod. The second hook is placed at a distance from the first one on the same vertebra or, if appropriate, on another vertebra. Such means are described in particular in document FR-A-2 816 196. In said document, the rod of small diameter is threaded and is inserted into a hole in the head of the hook which bears the connector. In document FR-A-2 767 263, the hook on which the spinal rod is fixed comprises, on its head, two channels: one in which a spinal rod is installed (therefore no connector is used here), the other in which a smaller rod is installed, which connects the hook to the other hook. The rods are immobilized in the head of the hook by threaded plugs. These means of fixation have the disadvantage of having relatively complex structures and of requiring, for their installation, a number of manoeuvres and a length of time which it would be desirable to reduce.

### BRIEF DESCRIPTION OF THE DRAWINGS

FIG. 1 is a perspective view of an embodiment of a spinal osteosynthesis device.

FIG. 2 is a perspective view of the embodiment of FIG. 1 connected with vertebral bone.

FIG. 3 is a perspective view of the embodiment of FIG. 1 including additional structure in a state of assembly.

FIG. 4 is a perspective view of the embodiment of FIG. 1 including additional structure in a further state of assembly.

FIG. 5 is a perspective view of the embodiment of FIG. 1 with an embodiment of an orthopedic longitudinal member connected thereto.

### DESCRIPTION OF THE DISCLOSED EMBODIMENTS

For the purposes of promoting an understanding of the principles of the invention, reference will now be made to the embodiment illustrated in the drawings and specific language will be used to describe the same. It will nevertheless be understood that no limitation of the scope of the invention is thereby intended. Any such alterations and further modifications in the illustrated device, and any such further applications of the principles of the invention as illustrated herein, are contemplated as would normally occur to one skilled in the art to which the invention relates.

As will be seen from the figures, an embodiment of a spinal osteosynthesis device comprises a first hook 1 intended to be attached to a vertebral body, such as at a lamina or opposite a pedicle of a vertebra. The head 2 of hook 1, in the illustrated embodiment of hook 1, supports a threaded protrusion or shaft 3 which is substantially perpendicular to upper surface 4 of the head 2. Upper surface 4 may be substantially flat. Hook I includes a blade 22, which is forked in the illustrated embodiment, connected to head 2. Hook 1 may also include one or more indentations 24, shown in the illustrated embodiment as adjacent upper surface 4. In a particular embodiment, two indentations 24 may be provided, one on each side of hook 1.

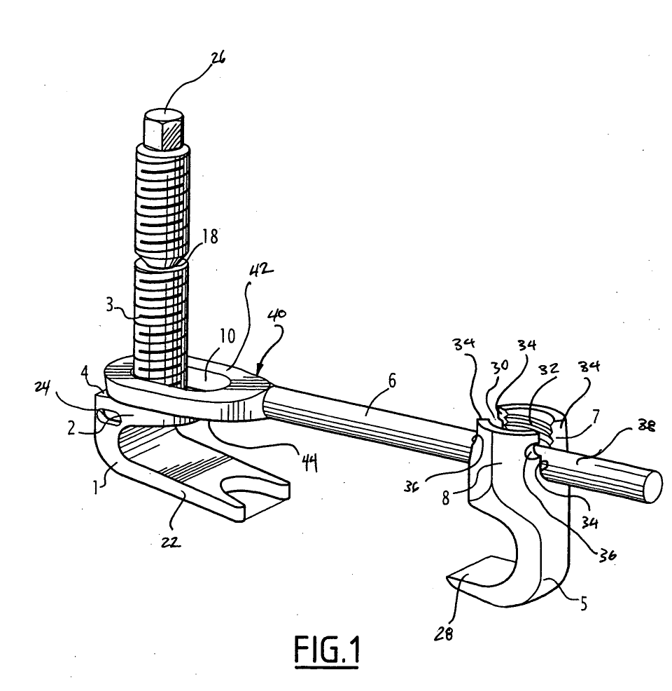

Shaft or protrusion 3 may be threaded as noted above or otherwise configured. If it is threaded, standard machine threads may be used, to accommodate a nut or other locking member. At a medial portion of the illustrated embodiment of shaft 3, a line of lesser resistance (e.g. weakened or small-diameter section) 18 is found. A head portion 26 may be placed on shaft 3. As shown in FIG. 1, head portion 26 can be placed at or near the top of shaft 3, and can be configured to accommodate a wrench or holding tool, as with the hexagonal configuration of the illustrated embodiment. Other configurations or placements of head portion 26 may be used. A second hook 5 is provided for connection to a vertebral body, such as a posterior arch or to a transverse process of a vertebra, either the same vertebra to which hook 1 is attached or, if appropriate, a different vertebra. The illustrated embodiment of hook 5 includes a blade portion 28, and a head 8 having a groove or channel 30 substantially parallel to blade 28 and internal threads 32 bordering channel 30. It will be understood that the channel 30 could have other angles with respect to blade 28, and threads 32 could be external, could be replaced by other surface configurations, or could be removed in other embodiments. As seen particularly in FIG. 1, in one embodiment head 8 of hook 5 may have side surfaces 34 adjacent channel 30 that are angled with respect to channel 30, to assist the surgeon in inserting items into channel 30 from the side. Head 8 may also have side indentations 36 adjacent channel 30, that can be configured to accommodate a gripping, holding or positioning tool or other structure.

As is known, hooks 1, 5 can be connected via a rod 6 that in the illustrated embodiment is of small diameter. The latter (i.e. rod 6), in the illustrated embodiment, has one of its ends 38 or a middle portion simply placed in groove 30 in the head 8 of the second hook 5, where it is free in translation and in rotation before being immobilized there by a threaded plug or set screw 9. In the illustrated embodiment, middle portion or end 38 of rod 6 is substantially cylindrical. The other end 40 of rod 6 is flattened (e.g. with substantially flat upper surface 42 and lower surface 44) in the illustrated embodiment and has an aperture 10, which may be oblong with a length of the order of 10 mm, for example. Shaft or protrusion 3 can be fitted through said aperture 10 in such a way that the flattened end 40 of the rod 6 rests on the head 2 of the first hook 1 (e.g. substantially flat lower surface 44 contacts substantially flat surface 4) and end 40 can be immobilized against head 2 by a locking member such as a nut, an example of which will be described further below. Rod 6 has a diameter generally of the order of 3 mm; it is referred to as being “of small diameter” above simply by contrast to the rod 13 which generally has a substantially greater diameter (of the order of 5.5 mm, for example).

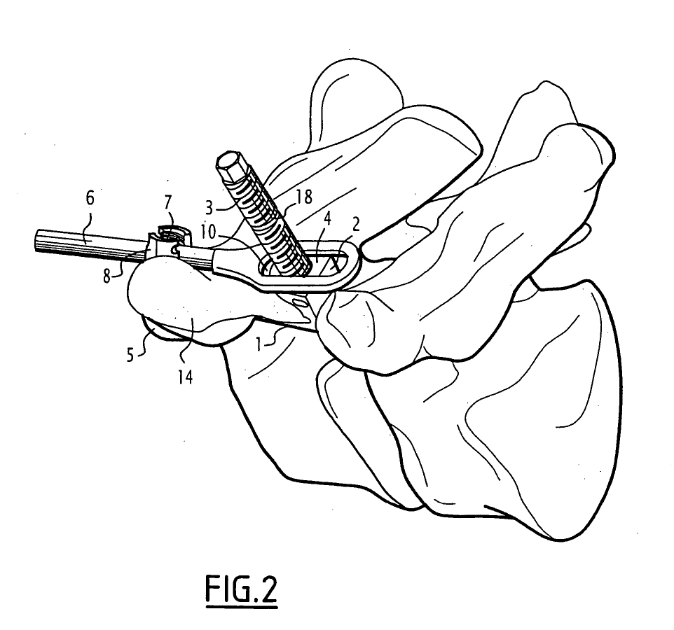

FIG. 2 shows an example of a stage involved in fitting the disclosed structure on a vertebra 14 of the patient. Hook 1 is installed opposite a pedicle of the vertebra 14, and hook 5 on the transverse process of the same vertebra 14, it being understood that this arrangement is just one example and that any other type of location of the hooks on the vertebra is contemplated. Rod 6 connecting the two hooks 1, 5 may be arranged as in FIG. 1. For example, aperture 10 of rod 6 may be placed over shaft 3 and against or near surface 4 of hook 1, and middle or end portion 38 of rod 6 may be placed in channel 30 of hook 5 by insertion through the top and/or side of channel 30. It will be noted that the oblong shape of aperture 10 allows the surgeon great latitude in positioning rod 6 in the best possible location without difficulty. Likewise, the angular and translational position of hook 5 can be established without difficulty.

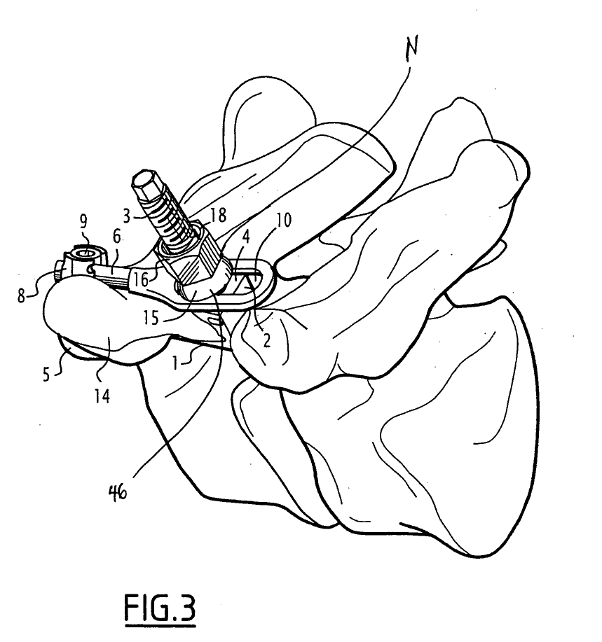

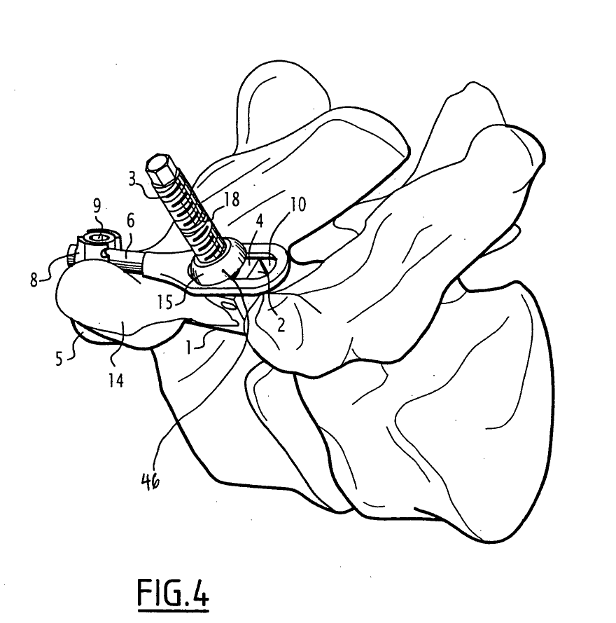

FIG. 3 shows an example of another stage of fitting the disclosed structure. Rod 6 of small diameter has been immobilized on the hooks 1, 5, on the one hand by the threaded plug 9 screwed into groove 30 in the head 8 of the second hook 5, and, on the other hand, by a nut N fitted on the protrusion 3. This nut N comprises two parts separated by a line of lesser strength (not visible in FIG. 4) which makes nut N divisible. The lower part 15 of nut N is designed with a conical (or spherical) upper surface 46 intended to cooperate with the connector 11, as discussed below. The upper part 16 has flat faces allowing nut N to be tightened by means of a suitable tool. Thus, when upper part 16 is tightened, lower part 15 is forced against upper surface 42 of rod 6. Applying sufficient torque to upper part 16 or cutting or applying other force at the line of lesser strength causes a break or rupture at or near the line of lesser strength, separating upper part 16 from lower part 15. In this way, upper part 16 can be removed while lower part 15 is maintained securely against upper surface 42 of rod 6. Before or after rod 6 is immobilized with respect to one or both of hooks 1, 5, it is preferable to cut off its part extending beyond the second hook 5 (e.g. at least part of end 38).

FIG. 4 shows an example of another stage of fitting the disclosed structure, in which stage the upper part 16 of nut N fitted on the protrusion 3 has been removed. All that remains of nut N, therefore, is its lower part 15 which is ready to receive the connector 11.

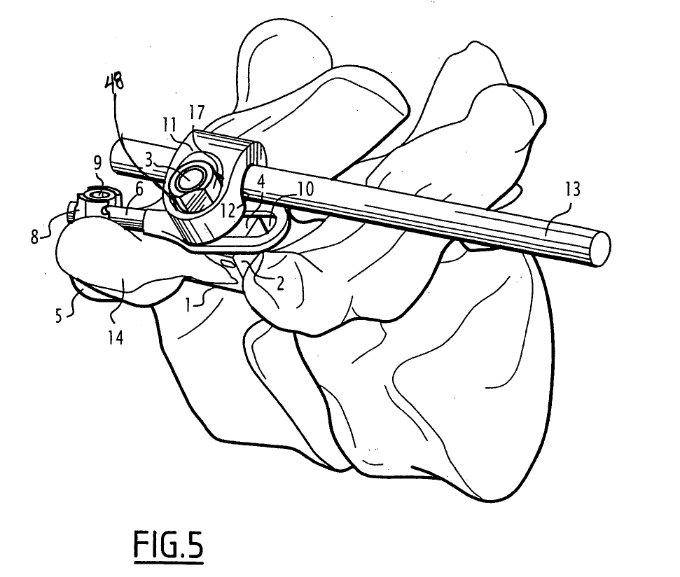

FIG. 5 shows an example of another stage of fitting the disclosed structure. In that embodiment, a spinal rod 13 has been connected to it. To do this, a connector 11 is provided. Connector 11 in one embodiment includes a longitudinal orifice 12 sized and shaped to accommodate rod 13 and an aperture 48. In the illustrated embodiment, orifice 12 has a longitudinal axis and is open below and/or to a side of that axis, so that rod 13 can be inserted into orifice 12 longitudinally and/or laterally with respect to that axis. Aperture 48 is sized and configured to allow at least part of shaft 3 to be inserted through it. In a particular embodiment, the height of connector 11 adjacent aperture 48 is such that line of lesser resistance 18 extends above aperture 48.

Connector 11, in whose longitudinal orifice 12 the rod 13 has first been fitted in one embodiment, is placed on the protrusion 3 and over lower part 15 of nut N, and then clamped by a nut 17 which has the dual function of clamping connector 11 on the remaining part 15 of nut N and of wedging the rod 13 inside the connector 11. In particular embodiments, rod 13 is held between a portion of connector 11 (e.g. a wall defining orifice 12) and the remaining part 15 of nut N, and/or between a portion of connector 11 (e.g. a wall defining orifice 12) and upper surface 42 of rod 6. After the nut 17 has been tightened, the part of the protrusion 3 emerging from it is, as is customary, broken or cut off by the surgeon along line of lesser resistance 18. The example which has just been described and shown is of course non-limiting, and various modifications can be made to it.

For example, the disclosed structure can be installed not on just one vertebra, but on two different vertebrae, in such a way as to form a sub-assembly of vertebrae to which the rod 13 is connected. The designs of the hooks 1, 5 can be modified depending on the site of installation envisaged. Further, the rod 6 can be connected to hook 5 by means other than those shown.

The configuration of connector 11 can be different, in which case the configuration of the nut N and/or parts 15, 16 clamping the rod 6 on the head 2 of hook 1 can be modified. It is not necessary for nut N or parts 15, 16 to be divisible.

In the example shown, end 40 of rod 6 with aperture 10 is flattened in one embodiment in order to cooperate with a planar embodiment of upper surface 4 of hook 1, but other designs are conceivable, for example a sphere-to-sphere contact between the rod 6 and the hook 1.

In the example shown, protrusion 3 is made in one piece with hook 1 and perpendicular to the latter's upper surface 4, but it can also be formed by a separate component connected to hook 1 in a way which allows protrusion 3 to be angled with respect to surface 4 of hook 1 inside a cone. This angulation, which is normally of the order of at most 30°, can make it easier to fit connector 11, rod 13 or other orthopedic parts because it permits approximate or preliminary bending of rod 13 prior to connection with connector 11. If the configurations of the means for connecting protrusion 3 to hook 1, and of nut N for immobilizing rod 6 on hook 1, so permit, this angulation can, if appropriate, persist after the construct has been immobilized. Means for ensuring this angulation are well known in the prior art, for example in document WO-A-02/38061, where they are applied to plates.

A spinal orthopedic construct by a posterior approach, with which the disclosed structures can be used, can, depending on the surgeon's preference and on its location on the spine, comprise only connection devices as disclosed herein, or simultaneously comprise one or more connection devices as disclosed herein and one or more connection devices of other types.

Thus, among other things apparatus is disclosed herein for connecting an orthopedic rod of a spinal osteosynthesis device to a vertebra or to two vertebrae, of which embodiments may include a first hook provided with a threaded protrusion for fitting a connector in which an orthopedic rod is inserted, a second hook, a rod connecting the two hooks, and means for immobilizing the ends of the connecting rod on the hooks. The end of the connecting rod in contact with the first hook may be an oblong aperture, and the means for immobilizing this end on the head of the first hook may be formed by a nut. The nut can be a divisible nut, a lower part of which can cooperate with the connector. The protrusion can have an angulation with respect to the upper surface of the first hook. An end of the connecting rod may be flattened so as to cooperate with an upper surface of the head of the first hook. The head of the second hook can comprise a groove for insertion of an end of the connecting rod, and the means for immobilizing said end in said head can be formed by a threaded plug. Also disclosed is a spinal osteosynthesis apparatus including a rod, connectors, and structure for joining said connectors to the patient's vertebrae, and can be fitted by, for example, a posterior approach.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. An apparatus comprising:
a first hook provided with a threaded protrusion;
a second hook;
a rod for connecting said hooks, said connecting rod having a first end in contact with said first hook, said first end including an oblong aperture, and a second end in contact with said second hook; and
means for immobilizing said ends of said connecting rod on said hooks, said means for immobilizing said first end on said first hook including a nut.

2. The apparatus of claim 1, further comprising a connector having at least one aperture for accommodating said protrusion and at least one aperture for accommodating a spinal rod.

3. The apparatus of claim 2, wherein said nut is a divisible nut having a lower part adapted to cooperate with said connector.

4. The apparatus of claim 3, wherein said lower part of said nut has an exterior surface that is at least part spherical, whereby said connector can pivot with respect to said lower part of said nut.

5. The apparatus of claim 1, wherein said first end of said connecting rod has a substantially flat lower surface, and said first hook has a substantially flat upper surface.

6. The apparatus of claim 5, wherein said second end of said connecting rod is substantially cylindrical.

7. The apparatus of claim 6, wherein said second end of said connecting rod is longer than said first end.

8. The apparatus of claim 1, wherein said first hook has an upper surface and said protrusion is angularly orientable with respect to said upper surface of said first hook.

9. The apparatus of claim 1, wherein said first hook has a planar upper surface and said first end of said connecting rod is flattened so as to cooperate with said upper surface of said head of said first hook.

10. The apparatus of claim 1, wherein said second hook has a head including a groove for insertion of an end of said connecting rod, and wherein said means for immobilizing said end in said head of said second hook include a threaded plug.

11. An apparatus comprising:
a spinal rod;
a connector on said spinal rod;
a first hook provided with a threaded protrusion, said protrusion connected to said connector;
a second hook;
a connecting rod for connecting said hooks, said connecting rod having a first portion in contact with said first hook, said first end including an oblong aperture, and a second portion in contact with said second hook;
a locking member adapted to lock said second portion of said connecting rod with respect to said second hook;
a first nut threaded onto said protrusion and locking said first portion of said connecting rod with respect to said first hook; and
a second nut threaded onto said protrusion and against said connector, forcing said connector against said first nut to lock said connector with respect to said connecting rod and said spinal rod.

12. The apparatus of claim 11, wherein said protrusion is multi-axial with respect to said first hook.

13. The apparatus of claim 11, wherein said first nut is a divisible nut.

14. The apparatus of claim 13, wherein said divisible nut has a lower portion and an upper portion, and said lower portion contacts said connector.

15. The apparatus of claim 14, wherein said lower portion has a curved outer surface.

16. The apparatus of claim 15, wherein said lower portion has an outer surface that is at least part spherical.

17. The apparatus of claim 14, wherein said connector is swivelable on said lower portion of said first nut.

18. An apparatus, comprising:
a spinal rod;
a first hook having a head, said head being provided with a threaded shaft;
a second hook;
an elongated member connecting said first and second hooks, said elongated member having a first end with an aperture therethrough adapted to contact said head of said first hook;
a connector having an orifice and an aperture, wherein at least a portion of said spinal rod is within at least a portion of said orifice, and at least a portion of said shaft is within at least a portion of said aperture; and
a nut for immobilizing said first end with respect to said head of said first hook, said nut adapted to cooperate with said connector.

19. The apparatus of claim 18, wherein said elongated member further includes a substantially cylindrical rod portion.

20. The apparatus of claim 18, wherein said elongated member has a longitudinal axis, and said aperture of said elongated member is oblong and is directed substantially along said axis.

21. The apparatus of claim 20, wherein said elongated member further includes a substantially cylindrical rod portion that extends substantially along said axis.

22. The apparatus of claim 21, wherein said rod portion of said elongated member is connected to said second hook.

23. The apparatus of claim 22, wherein said second hook includes a head having a channel and at least a part of said rod portion of said elongated member is within said channel, and further comprising a locking member connected to said second hook in said channel to lock said part of said rod portion of said elongated member within said channel.

24. The apparatus of claim 18, wherein said second hook includes a head having a substantially U-shaped channel extending from a first end to a second end of said second hook, said channel having a longitudinal axis, said second hook having at least one substantially planar surface adjacent said channel that is oblique to said axis.

25. The apparatus of claim 18, wherein said shaft includes a medial portion having a line of lesser resistance, said line extending above said connector.
